(19) United States
(12) Patent Application Publication
(10) Pub. No.: US 2003/0017195 A1
(43) Pub. Date: Jan. 23, 2003

(54) Method for oral drug delivery

(76) Inventors: Samir Mitragotri, Goleta, CA (US); Zancong Shen, Goleta, CA (US)

Correspondence Address: Samir Mitragotri, 6831 Sweetwater Way, Goleta, CA, 93117, US

(21) Appl. No.: 10/199,355

(22) Filed: Jul. 18, 2002

Related U.S. Application Data
(60) Provisional application No. 60/307,059, filed on Jul. 20, 2001.

(51) Int. Cl. A61K 9/70; A61K 9/16; A61K 9/50

(52) U.S. Cl. 424/449; 424/490

(57) ABSTRACT

A novel mucoadhesive patch system for drug delivery comprising an impermeable backing layer, a drug reservoir and a mucoadhesive layer. After introduction into the gastrointestinal tract, the mucoadhesive layer of the patch sticks to the lumenal wall, then the drug releases from the reservoir in a unidirectional way through the mucoadhesive layer into intestine mucosa. The patch system and method of drug delivery is advantageous in enhancing bioavailabilities of poorly absorbed drugs such as polar molecules or bioactive peptides and proteins.

This application claims the benefit of provisional application No. 60/307,059 filed Jul. 20, 2001, which is incorporated herein by reference.

### FIELD OF THE INVENTION

The invention relates generally to the field of drug delivery and more specifically to the fabrication of patches for oral delivery of therapeutic agents

### BACKGROUND OF THE INVENTION

Oral route has attractive advantages for drug delivery including ease of application and high patient compliance. However, for poorly absorbed molecules and enzyme-sensitive bioactive agents particular strategies are required to achieve sufficient drug absorption into the blood circulation. Several modifications of simple dosage systems including, liposomes (J. Okada, S. Cohen and R. Langer, In vitro evaluation of polymerized liposomes as an oral drug delivery system. Pharm. Res. 12 (1995), pp. 576-582 and H. Chen, V. Torchilin and R. Langer, Polymerized liposomes as potential oral vaccine carriers: stability and bioavailability. J. Controlled Release 42 (1996), pp. 263-272.); mircoparticles (Mathiowitz, J. S. Jacob, Y. S. Jong, G. P. Carino, D. Chickering, P. Charturved, C. A. Santos, K. Vi jayaraghavan, S. Montogomery, M. Bassett and C. Morrell, Biologically erodable microspheres as potential oral drug delivery systems. Nature 386 (1997), pp. 410-414 and N. Santiago, S. Milstein, T. Rivera, E. Garcia, T. Zaidi, H. Hong and D. Bucher, Oral Immunization of rats with proteinoid microspheres encapsulating influenza virus antigens. Pharm. Res. 10 8 (1993)); and nanoparticles (C. Damgé, C. Michel, M. Aprahamian and P. Couvreur, New approach for oral administration of insulin with polyalkycyanoacrylate nanocapsules as drug carrier. Diabetes 37 (1988), pp. 246-251; Carino GP, Jacob JS, Mathiowitz E. Nanosphere based oral insulin delivery. J Control Release 2000 Mar. 1;65(1-2):261-9; and A. M. Hillery, I. Toth and A. T. Florence, Co-polymerised peptide particles II: Oral uptake of a novel copolymeric nanoparticle delivery system for peptides. J. Controlled Release 42 (1996), pp. 65-73) have been used as drug carriers to overcome the poor drug bioavailibility.

Particular attention has been paid to mucoadhesive micro/nanoparticles that adhere to intestine mucus and therefore prolong their migration time and extend release of the drug (H. Chen, V. Torchilin and R. Langer, Lectin-bearing polymerized liposomes as potential oral vaccine carriers. Pharm. Res. 13-9 (1996), pp. 1378-1383; Kawashima Y, Yamamoto H, Takeuchi H, Kuno Y. Mucoadhesive DL-lactide/glycolide copolymer nanospheres coated with chitosan to improve oral delivery of elcatonin. Pharm Dev Technol 2000; 5(1):77-85; and Lim S T, Martin G P, Berry D J, Brown M B. Preparation and evaluation of the in vitro drug release properties and mucoadhesion of novel microspheres of hyaluronic acid and chitosan. J Control Release 2000 May. 15;66(23):281-92). However, several issues limit applicability of these particle systems. Specifically: i) drug release is not unidirectional, therefore certain fraction would get lost into the lumenal fluid and ii) since the particle surface is exposed to the intestine fluid, bioactive agents encapsulated in these particles may not get sufficient protection from proteolytic degradation in the intestine.

### BRIEF SUMMARY OF THE INVENTION

This invention discloses a novel intestinal mucoadhesive patch system for oral drug delivery. The patch system comprises an impermeable backing layer, a drug reservoir and a mucoadhesive layer. The drug reservoir and the mucoadhesive layer may be combined into a single layer. When the patches are introduced into the gastrointestinal tract, the mucoadhesive layer sticks to the lumenal wall due to it's mucoadhesive properties, then the drug releases from the reservoir in a unidirectional way through the mucoadhesive layer into the intestine mucosa. This improved method is advantageous in enhancing bioavailabilities of poorly absorbed drugs such as polar molecules or bioactive peptides and proteins.

The present invention provides several significant advantages over conventional oral delivery systems described in the art, including, 1) The backing layer of this patch prevents drug from leaking into the outer lumen and induces a unidirectional release of drug into epithelial layer. This unidirectional release characteristic results in an increase in the local drug concentrations, which may accordingly enhance the absorption efficiency. 2) A patch sticking on lumenal wall by mucoadhesive layer extends transit of drugs in intestine, resulting in a sustained release behavior. 3) For bioactive agents such as peptides or proteins, protection of these agents by this patch system would reduce the chance of proteolysis.

The patch system of the present invention would also become a potent delivery system for bioactive agents such as peptides and proteins. The present invention could potentially protect these molecules from proteolytic degradation in intestine thereby increasing their oral bioavailability. As more peptides and proteins drugs emerge into the market, this novel invention would become an excellent delivery system to enhance oral delivery of poorly absorbed drugs as an alternative approach for invasive administration.

### DEFINITION OF TERMS

Patch: A patch is a disk-shaped object constructed from biocompatible materials whose lateral dimension is substantially higher than the transverse dimension. Typical diameter of the patch described here is between 500 micrometer and 5 millimeter. The thickness of the patch is between 100 and 1000 micrometer.

Adhesion: Adhesion of patches on intestinal wall is defined as the action of holding the patch on the intestinal membrane without requiring an external force.

Capsule: A capsule is a hollow containment that can be filled with patches. A capsule is also considered as a type of containment.

Mucoadhesion: Mucoadhesion is the adhesion of patches on the mucous layer of the intestine.

### BRIEF DESCRIPTION OF THE DRAWINGS

FIG. 1 shows schematic descriptions of the microsphere-based mucoadhesive patch system of the present invention.

FIG. 2 shows schematic descriptions of the compressed mucoadhesive patch system of the present invention.

FIG. 3 shows an oral tablet in which patches are incorporated. FIG. 3A shows an oblique view. FIG. 3B shows a cross section.

FIG. 4 shows a capsule in which patches are incorporated. FIG. 4A shows an oblique view. FIG. 4B shows a cross section.

FIG. 5 illustrates unidirectional diffusion of model drug sulforhodamine B from mucoadhesive (▪) or backing layer side (⋄).

FIG. 6 shows the drug released after 12 hours from either side of the patch

FIG. 7 shows the transport of sulforhodamine B across rat intestine in (▪) patch system or (∘) solution form.

FIG. 8 shows the transport of phenol red across rat intestine in (▪) patch system or (∘) solution form.

FIG. 9 shows bioavailability of sulforhodamine B across intestine at each time point from intestinal patches and solution.

FIG. 10 shows bioavailability of phenol red across intestine at each time point from intestinal patches and solution.

FIG. 11 shows an image of patches. Figure 11A shows an image of a microsphere-based patch and Figure 11B shows an image of a compressed patch (2 or 4 mm in diameter, 400 μm in thickness). The patch consists of a backing layer (Ethylcellulose) coating all but one faces of the drug reservoir. The drug reservoir is composed of drug and mucoadhesive hydrogels.

FIG. 12: Release of sulforhodamine B from patches into the lumen and intestinal membrane,

FIG. 13 shows adhesion force between the patch and the pig intestine mucosa measured in the intestinal loop. Effect of PBS in the intestine on the adhesion force of the patch: compressed patch (▪), non-compressed patch (ο).

FIG. 14 shows adhesion force between the patch and the pig intestine mucosa measured in the intestinal loop. Effect of contact time on the adhesion force of the patch: compressed patch (▪), non-compressed patch (ο).

FIG. 15 shows adhesion of patches on intestinal mucosa with different thicknesses of PBS layer. Adhesion forces of the patch on intestinal mucosa: compressed patch (▪) and non-compressed patch (●).

FIG. 16 shows an image of a patch adhered to intestinal mucosa.

FIG. 17 shows blood glucose levels after intestinal delivery of insulin-loaded patches in non-diabetic rats: 5 IU/kg insulin patch (▪), 10 IU/kg insulin patch (♦), 5 IU/kg insulin 10 mg sodium glycocholate (□), Blank patch without insulin (ο), 10 IU/kg insulin solution pH 7), (▴) and 1.0 IU/kg insulin solution (pH 7) by subcutaneous injection (●). Error bars=SD, n=3-5

FIG. 18 shows the effect of chemical enhancers incorporated into the patch on drug transport.

FIG. 19 shows release of patches from capsule and adhesion to intestinal membrane.

### DETAILED DESCRIPTION OF THE INVENTION

Oral route has attractive advantages especially for improving the compliance of patients. However, for poorly absorbed molecules, enzyme-sensitive bioactive agents or drugs that require site-specific targeting delivery, particular strategies are needed to achieve sufficient drug absorption into the blood stream. In the foregoing conventional methods, particles such as liposomes, micro/nanoparticles or micro/nanocapsules are used as drug carriers to overcome the poor bioavailabilities of these drugs. Additionally, by coating mucoadhesive polymers on their surface, these particles can easily adhere to intestine mucus and therefore prolong their migration time and an extend release of the drug.

There are some limitations to these particle systems. Specifically, 1) Drug release is not unidirectional, therefore certain fraction would get lost into the lumenal fluid; 2) Transit of particles in the GI tract would cause high variability; and 3) As the particle surface is exposed to intestine fluid, bioactive agents encapsulated in these particles won't get sufficient protection from proteolytic degradation.

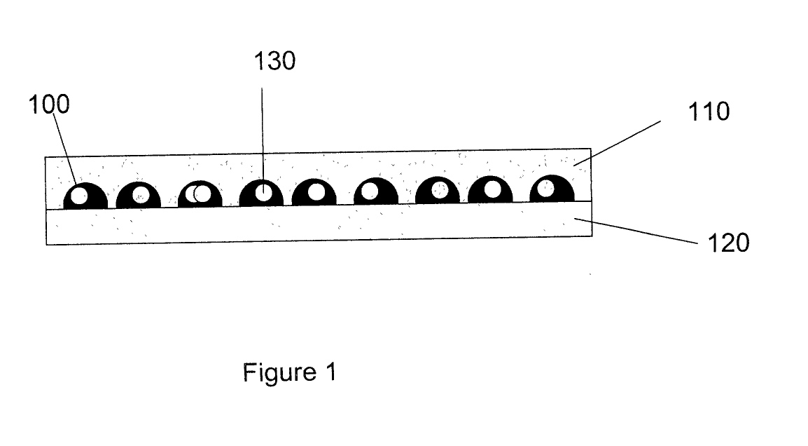

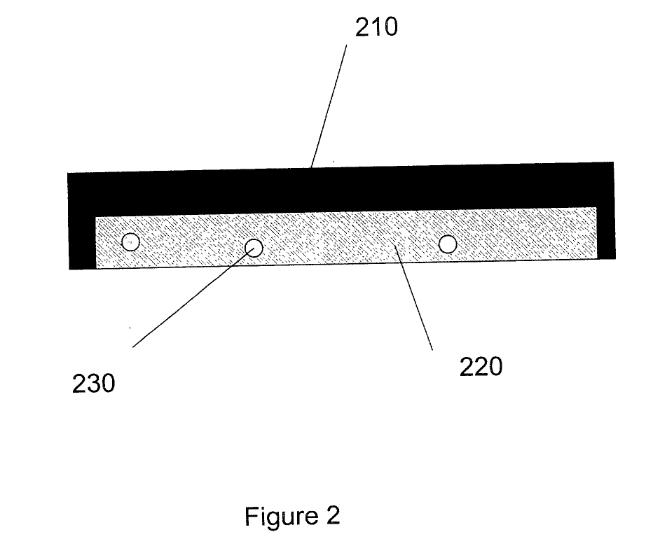

The present invention has solved or eliminated the above problems associated with particles as drug carrier. As shown in FIG. 1, the present invention is a patch system consists of 2 layers, a backing layer made of poorly permeable material such as ethyl cellulose (EC) or poly(lactide-co-glycolide) (PLGA) and mucoadhesive layer made of Carbopol, pectin, chitosan, SCMC, HPMC or other mucoadhesive polymers also containing the drug to be delivered. In the following, two types of patch designs are described. In the first design, the drug is first encapsulated in microspeheres and then embedded in the mucoadhesive layer. This design is referred to as a “microsphere-based patch” (FIG. 1). In the second design, the drug and the mucoadhesive material is mixed and compressed into a uniform film. This design is referred to as “compressed patch” (FIG. 2). Design of a microsphere-based patch is described first.

### DETAILED DESCRIPTION OF THE DRAWINGS

The drawings included herein show the design of patches for oral drug delivery. FIG. 1 shows the design of a microsphere-based patch. The patch consists of a layer of microspheres [100]. The microspheres may be prepared from a biocompatible material selected from the group including but not limited to: proteins, polysaccharides, polyanhydrides, polyesters, cellulose, and cellulose derivatives. The drug to be delivered [130] is incorporated into the microspheres. The drug can be selected from a group including but not limited to proteins, polysaccharides, and small molecules. Microspheres can be prepared using emulsion polymerization, spray drying, or vibrating nozzles. The layer of microspheres is spread on a mucoadhesive layer [120]. This layer is prepared from a mucoadhesive polymer including but not limited to Carbopol, pectin, and chitosan. The microsphere layer is covered with a poorly permeable polymer [110]. This polymer can be selected from a group including but not limited to poly-lactic co glycolic acid, and ethyl cellulose.

FIG. 2 shows a design of a compressed patch. The patch consists of a mixture of drug [230] and a mucoadhesive polymer [220] compressed to form a thin film. The drug can be selected from a group including but not limited to proteins, polysaccharides, and small molecules. The mucoadhesive layer may be prepared from a mucoadhesive polymer including but not limited to Carbopol, pectin, and chitosan. The compressed layer is covered with a poorly permeable polymer [210]. This polymer can be selected from a group including but not limited to poly-lactic co glycolic acid, and ethyl cellulose.

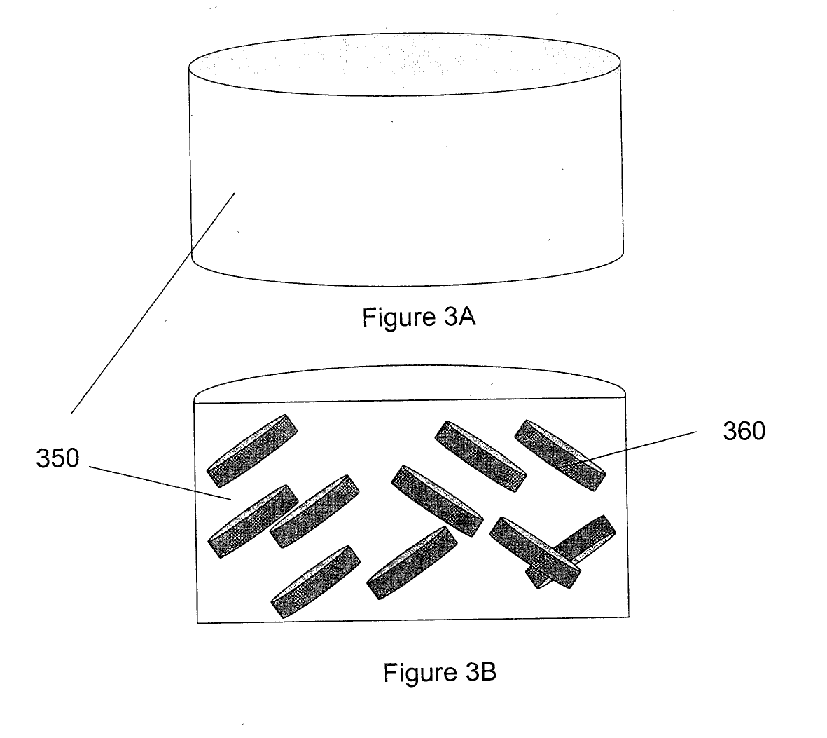

FIGS. 3A and 3B show a tablet [350] containing the patches [360]. This tablet is repared by mixing patches shown in FIGS. 1 and 2 and mixing them with a filler material. The filler material is an inert substance that does not react with the drug. An example of the filler material is lactose. The mixture of patches and the filler material is compressed to make a tablet shown in FIG. 3A.

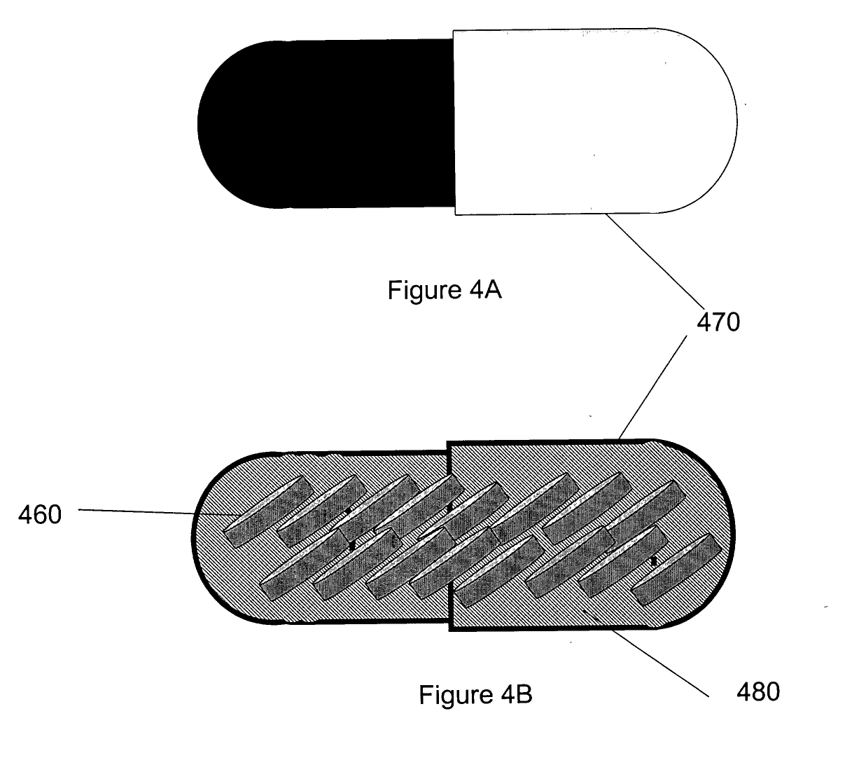

FIG. 4 shows a capsule containing patches. FIG. 4A shows an external view of the capsule. FIG. 4B shows a cross-section of the capsule. The capsule comprises a hollow containment made from a soluble material such as cellulose. The capsule may be coated with an enteric coating polymer [470] that allows the capsule to remain insoluble in the stomach but allows it to dissolve in the intestine. The capsule is filled with patches [460] and a filler material [480].

### Example 1

### Microsphere-Based Patches

(1) Formulation of microsphere-based patches: To formulate the patch system of the present invention, albumin microspheres (10-30 μm) containing hydrophilic drugs were made of Bovine serum albumin by an emulsion-crosslinking method. In this method, a 25% w/v aqueous BSA solution was dispersed in mineral oil at a speed of 1500 rpm. Microspheres were loaded with three different model drugs, sulforhodamine, phenol red, and FITC-dextran (MW 70,000 Da) in separate batches. In each case, an aqueous solution of these solutes was added to BSA solution prior to dispersing it in mineral oil. 100 μl of an aqueous solution of glutaraldehyde (25% v/v, Sigma Chemicals) was added to the emulsion and BSA was allowed to cross-link for 2 hours. Cross-linked BSA microspheres were washed first in petroleum ether, then in ethyl ether, and finally in acetone. This procedure produced uniform microspheres of size in the range of 10-30 μm.

To make a patch, mucoadhesive material such as Carbopol 934 (manufactured by B. F. Goodrich) or chitosan was first dissolved in water and then cast evenly on a Teflon plate (Teflon is manufactured by DuPont, U.S.A.). After drying, the mucoadhesive film was formed. Drug loaded (Sulforhodamine B) microspheres coated with Carbopol or carboxylmethylcellulose hydrogel were then spread uniformly on this mucoadhesive layer. Finally, microspheres were covered with a water impermeable material, such as EC or PLGA. The coating of microspheres by hydrophilic polymer (such as Carbopol or carboxymethylcellulose) plays an important role in opening a pathway for drug diffusing through the mucoadhesive layer. The original film can be cut into different shapes to become patches. The patch size may vary from 2-10 mm².

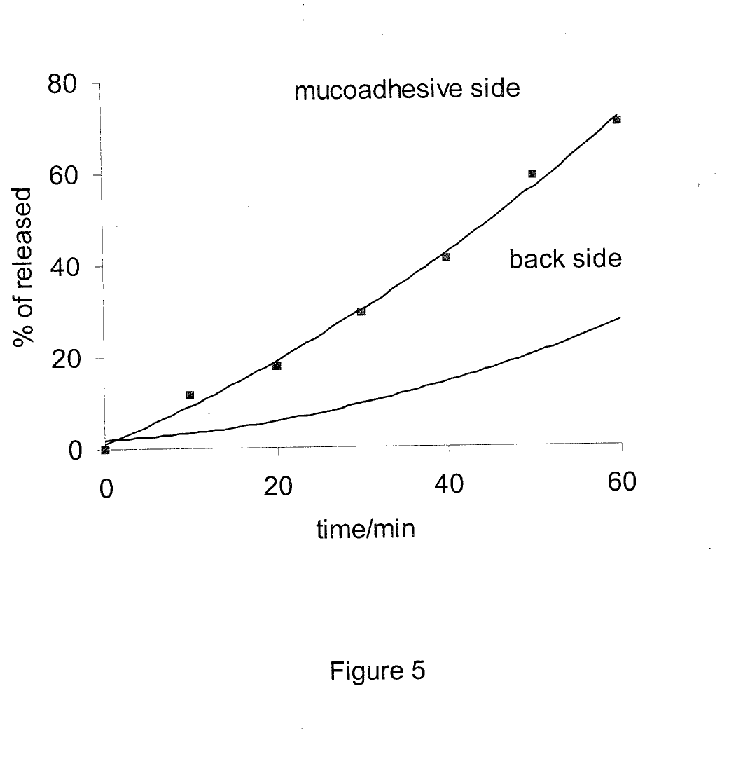

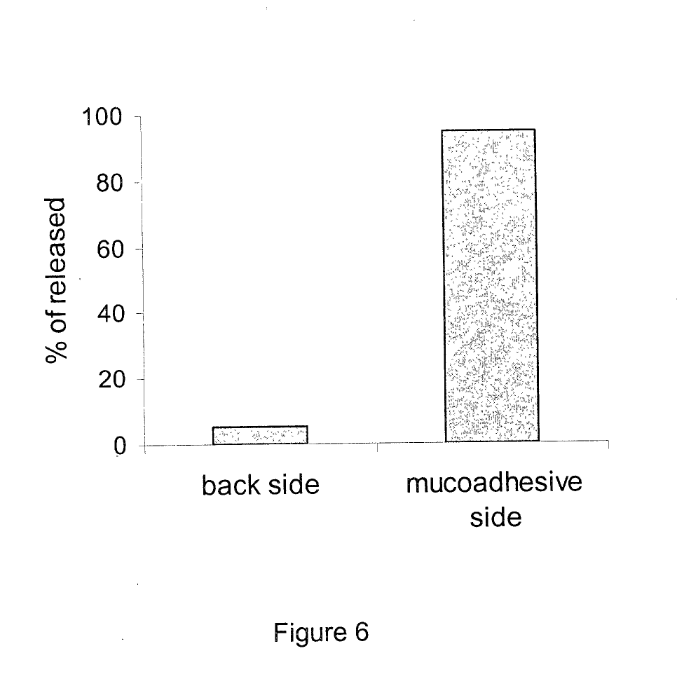

(2) Characterization of the microsphere-based patch system: In vitro release experiments reveal that this patch system exhibits a unidirectional drug diffusion behavior. More fraction of sulforhodamine B was observed to come out from mucoadhesive layer than the backing layer in either 60 minutes (FIG. 5) or 12 hours (FIG. 6). In another experiment, the mucoadhesive side of this patch was put on top of the mucosal layer of rat intestine, the back of this patch was covered by another intestine piece. After being immerged in PBS for 10 minutes, the intestine pieces were removed and the marks of sulforhodamine B on both intestine pieces were observed under microscope. More sulforhodamine B was found from the patch's mucoadhesive side. From these two experiments, it's proved that the fraction of drug that diffuses into intestinal mucosa from the patch's mucoadhesive layer is much greater than that from the backing layer. The intensity of red color is proportional to the amount of sulforhodamine B. This unidirectional diffusion increases local drug concentration in the absorptive epithelial layer.

Since microspheres in this patch were covered by the impermeable backing layer on both apical and lateral sides, this structure possesses a strong ability to retard drug leakage from the patch edges. In a regular patch without the microspheres structure, when the patch is cut into smaller pieces, drug in the reservoir layer easily leaks from along the breaking edges. In the presence of the microspheres structure, the breakpoint usually occurs between mircrospheres, which make it difficult for the drug to penetrate through the backing layer. So, with the help of microspheres structure, the leakage of drug from the patch can be substantially controlled. Though drug could leak from those microspheres along the edge, where their coating would possibly be broken, for microspheres located away from the edge no leakage can be seen. In a quantitative measurement, within 10 minutes, 60% of sulforhodamine B was lost from the edges in patch.without microspheres structure, while only 10% leaked in the presence of this microspheres structure. These experiments show that this unique structure guarantees significantly less leakage of drugs from the patch edges.

(3) In vitro absorption test of microsphere-based patch system: To investigate whether the mucoadhesive patch system of the present invention would have any enhancing effect for drugs transport across intestine, we selected small moleculesulforhodamine B, poorly absorbed drug- phenol red, and large molecule FITC-Dextran (MW=70,000) for the experiments. The experiment was performed in an in vitro perfusion device.

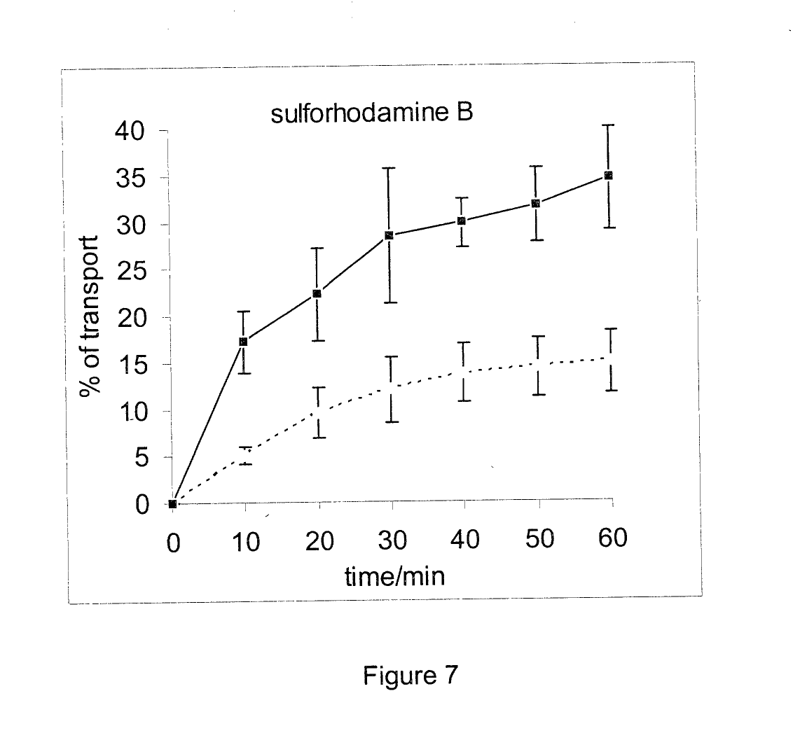

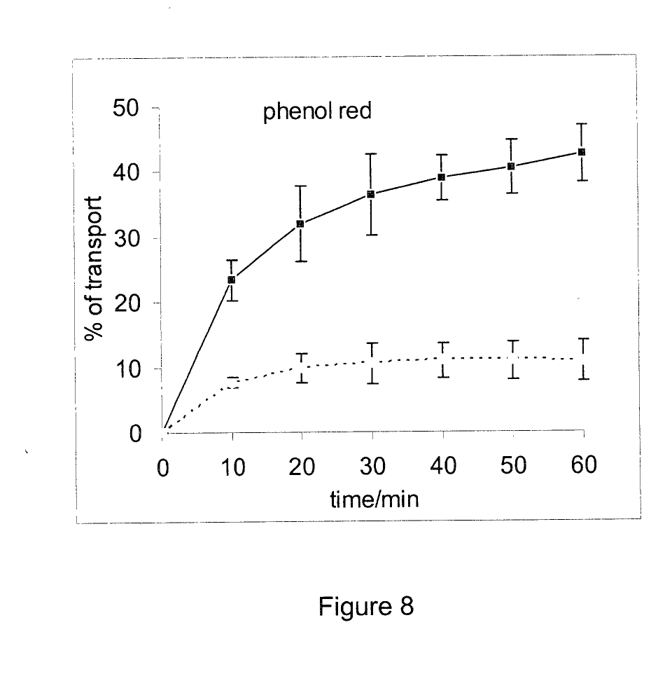

Patches (2 mm×2 mm) were put into intestine lumen (3 cm in length). One end of the lumen was connected to an infusion inlet, while the other end was connected to a receiving tube. Phosphate buffer solution (PBS) was infused in a constant rate (0.05 ml/min) into the lumen. The entire device was placed on a magnetic stirrer panel, and samples were taken from PBS outside the lumen at predetermined time interval. Quantitative measurement of drug concentration was conducted by spectrophotometry at 565 nm for sulforhodamine B and 560 nm for phenol red. This perfusion system mimics in-vivo intestine fluid movement. It was observed that compared to their solution form (∘), a higher fraction of sulforhodamine B (FIG. 7) or phenol red (FIG. 8) was transported across rat intestine from patches (▪). It was also noticed that the patch sticking on the intestine wall didn't fall off by the constant flow, hence the significant enhancement of these drugs' absorption is due to the mucoadhesiveness of the patch and unidirectional drug release characteristic.

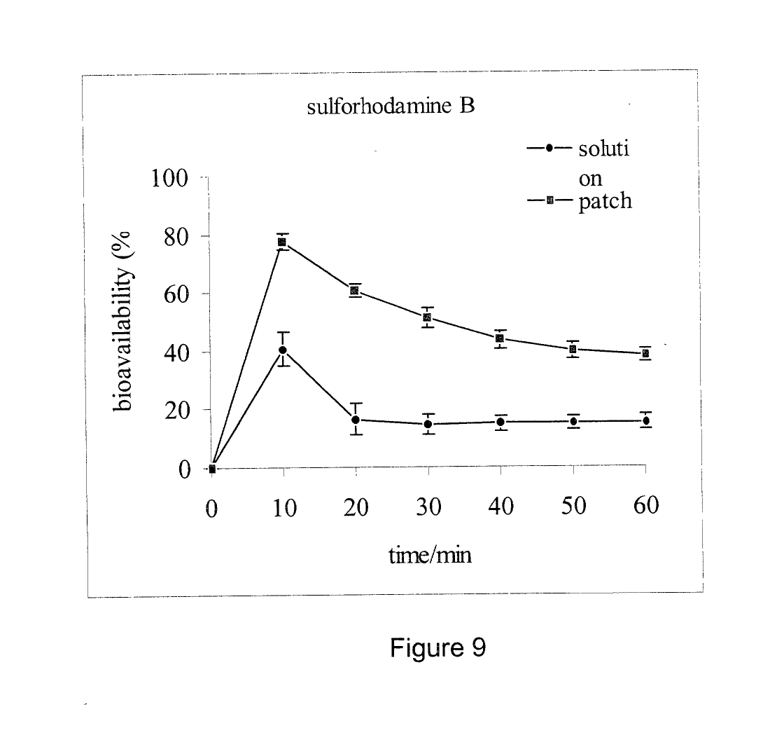

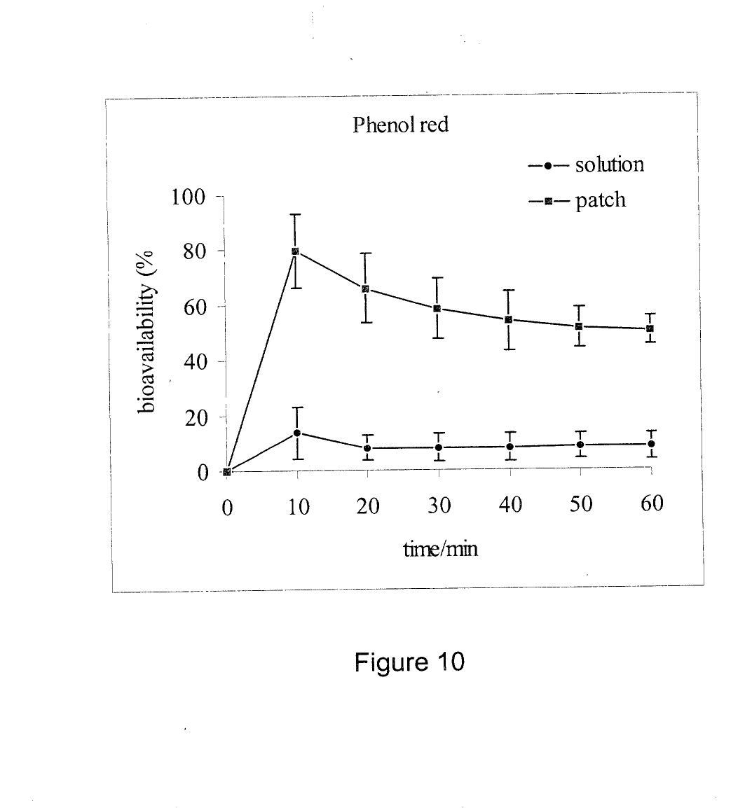

Bioavailability of drug transport from these patches was calculated. Bioavailability refers to the ratio of the amount of drug transported across the intestine to the total amount of drug released at the site of absorption. FIGS. 9 and FIG. 10 depict this bioavailability of sulforhodamine B (FIG. 9) and phenol red (FIG. 10) at different time points. As shown in the sulforhodamine B plot, after 10 minutes, approximately 80% of the drug was transported across the intestine from the patch (bioavailability is 80%), while in solution form, the bioavailability is only 40%. In the case of phenol red, the bioavailability is 80% for the patch and only 15% for the solution. This mucoadhesive patch system provides more fraction of the drug transported across the intestine layer.

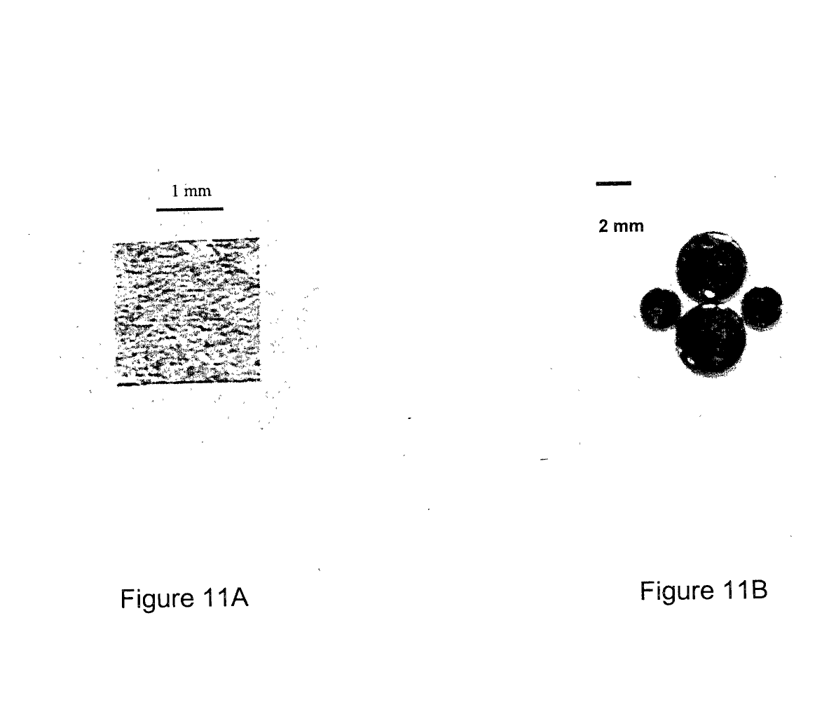

FIG. 11A shows an image of a microsphere-based patch.

### EXAMPLE 2

### Compressed Patches

(1) Formulation of compressed patches: To fabricate intestinal patches using compressed mixture of mucoadhesive polymers, a mixture of mucoadhesive powders Carbopol 934 (B F Goodrich Co. Cleveland, Ohio), pectin (Sigma Chemicals, St. Louis, Mo.), and sodium carboxylmethylcellulose (SCMC) (Carbopol: pectin: SCMC=1:1:2) was first prepared. Bovine insulin (MW=5733, 28.3 IU/mg, Sigma Chemicals, St. Louis, Mo.) was added to this mixture such that insulin concentration in the patch was 0.2-1.0 IU/mg. In some mixtures sulforhodamine B (Sigma Chemicals, St. Louis, Mo.) was added at a concentration of 10 μg/mg. 50 mg of the mixture was compressed under 1˜4 tons pressure using a hydraulic press (Carver Inc. Wabash, Ind.). This produced a 400 micrometer thick disk of a typical diameter of 13 mm. This disk was cut into smaller disks using a punch to produce disks possessing radii in the range of 2-4 mm. This disk was placed on a support and coated on all but one sides using a solution of Ethylcellulose (EC, Sigma Chemicals, St. Louis, Mo.) in acetone (20 mg/ml). Acetone was evaporated at room temperature. This procedure produced a thin layer of EC of about 50 micrometer. The resulting patches are shown in FIG. 11B.

(2) Unidirectional Release of Drug from Compressed Patches: Release of a model drug (Sulforhodamine B) from patches was measured in vitro into phosphate buffered saline (PBS, pH 7.4, 0.01 M). To distinguish drug release from the mucoadhesive side and the backing side of the patch, the patches were placed in a custom-designed diffusion cell. The cell comprised two chambers placed side-by-side with an opening provided between the chambers of about 3.14 mm². A patch (4 mm in diameter) was placed between the two chambers and each chamber was filled with 6 ml PBS. Vacuum grease was used to seal the joint to avoid leakage of PBS. Amount of sulforhodamine B released from either side of the patch into the solution was quantified at 565 nm using a spectrophotometer (UV-1601, Shimadzu Corporation).

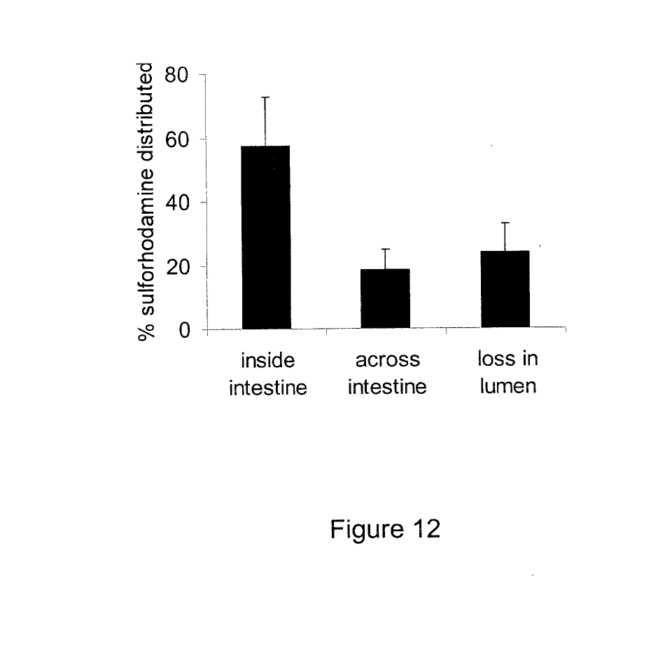

To assess whether unidirectionality of release is also observed when the patch is placed on the intestine, the following experiments were performed. A section of pig intestine was placed on a diffusion cell such that the mucosal side faced the upper (donor) chamber. The donor chamber was filled with 2 ml PBS. Under these conditions, the thickness of the PBS layer on the intestine was about 2 cm. A sulforhodamine-containing patch (4 mm in diameter) was prepared using methods described above was gently placed on the intestine. A stirring bar was placed on a mesh, which was placed about 1 cm above the patch. The receiver chamber was filled with 12 ml PBS. A stirring bar was placed in the receiver chamber. The cell was placed on a magnetic stirrer and stirred at 400 rpm for 120 minutes. Amount of sulforhodamine B released into donor and receiver chambers was measured using the same spectrophotometer described above. Percentages of sulforhodamine B delivered into the receiver and donor chambers were calculated (Figure 12). By measuring the total amount of sulforhodamine B released from the patch, the percent of sulforhodamine B delivered into the intestine was also calculated.

While about 10% of drug is released from the mucoadhesive side of the patch in 120 minutes, less than 0.3% is released from the backing side, indicating significant unidirectional release of drug from the patch,(more than 97% of drug was released from the mucoadhesive side). This was attributed to the impermeability of the backing layer. There was no significant difference in the release profile of sulforhodamine B when different compression pressures were used during patch preparation.

(3) Adhesion Force Measurement: Experiments were performed to determine the adhesion force between the patch and the intestine. The adhesive force is likely to depend on the patch characteristics, intestinal fluid content, the method of patch attachment and the method of measurement. To ensure that the measured adhesive force is not an artifact of any particular experimental method, we used two methods as described below.

Measurements in Intestinal Loops under Dynamic Conditions: These measurements are intended to mimic adhesion forces that may be observed in vivo. Freshly harvested small intestine (Yorkshire pigs) was used in these studies. The intestine was rinsed with 100 ml PBS and then cut into 5 cm long loops. One end of the loop was tied off and different volumes of PBS (0.5, 1.0, 2.0, 3.0, 4.0 ml) were added to the lumen. 8-10 intestinal patches (4 mm in diameter and 400 μm thick) were randomly inserted in the intestine loop. The other end of the loop was also tied off. The whole intestine loop was placed on a rocker (Boekel Scientific, Feasterville, Pa,) and shaken for 1 hour. The intestine was carefully cut open, a plastic cylinder (2 mm in diameter, 20 mm in length) was glued onto the backing layer of the patch using minimal amount of cyanoacrylate (Sigma Chemicals, St. Louis, Mo.). The whole intestine piece was then fastened on a bench balance (0.01 g resolution, Mettler Toledo, Columbus, Ohio.). The rod was gradually elevated at a rate of about 2.0 mm/s using a pulley until the patch detached from the intestine. The mass recorded by the balance during patch detachment was acquired and detachment force per unit patch area was calculated. To assess whether the adhesion force of the patch is time-dependent, patches were inserted in the intestine loop filled with a fixed volume (1.0 ml) of PBS and incubated for 0.5, 1.0, 2.0, 3.0 or 4.0 hours. At the end of the incubation period, patch detachment force was determined using methods discussed above.

Measurements using Planar Intestine Samples under Static Conditions: These tests are intended to measure the adhesion of patches under submerged conditions. For this purpose, a pig intestine loop was cut open and placed on a custom-built glass chamber (15 mm in diameter, 19 mm high) with the mucosal side facing up. The chamber was then filled with various amounts of PBS (29.6, 59.2, 148, 296, 592 or 1184 μl, corresponding to 0.17, 0.34, 0.84, 1.68, 3.35 or 6.7 mm thickness of PBS layer). Patches (with or without compressing procedure) (4 mm in diameter and 400 μm thick, 3-4 pieces) were gently placed on the mucosal surface with the mucoadhesive side facing the mucosal side of the intestine. For a PBS thickness up to 0.34 mm, patches were only partially submerged under PBS. Beyond this thickness, the patches were completely submerged under PBS. After 1 hour, PBS inside the chamber was removed and the measurement of adhesion force of the patches was carried out using the method described above. To assess the significance of a particular thickness of PBS layer on the intestine, one could estimate the volume percent occupied by PBS using the thickness of PBS on the intestine and the diameter of the pig small intestine (20 mm). A PBS thickness of 0.17, 0.34, 0.84, 1.68, 3.35 or 6.70 mm corresponds to a volume percent of 2.7, 5.3, 13.0, 25.0, 46.4 or 78.5 respectively.

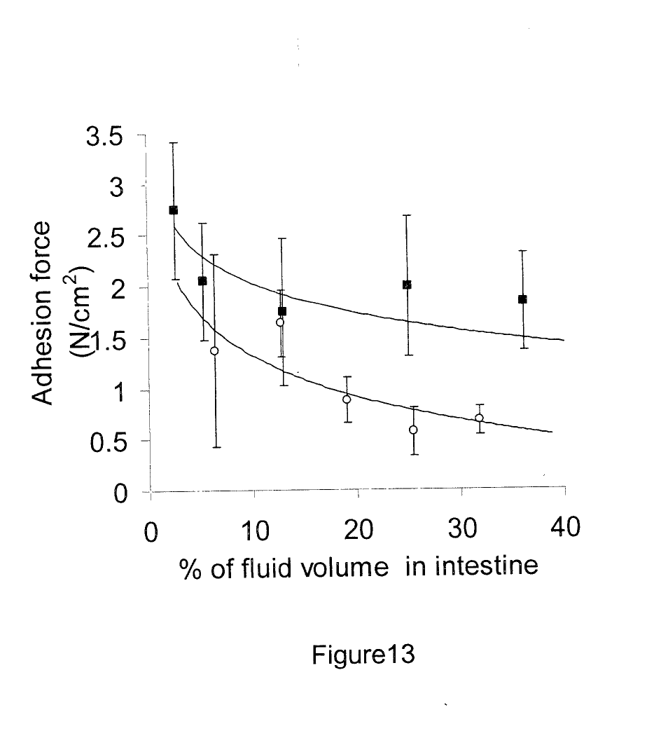

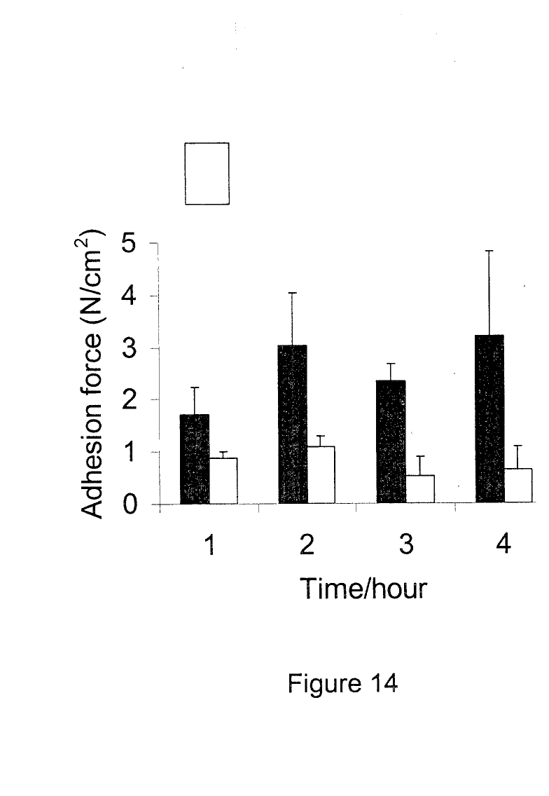

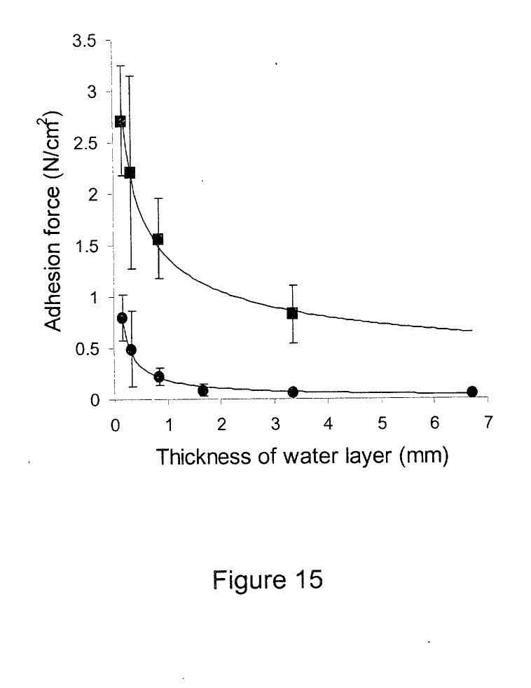

The patches randomly inserted into the intestinal loops adhered well to the lumenal wall. After one hour of incubation, about 87% of patches were found attached to the lumenal wall by their mucoadhesive sides (data not shown). No patches attached by the backing (ethylecellulose) layer. The adhesion force ranged from 1.5-3 N/cm² and was nearly independent of time over a period of 4 hours (filled bars, FIG. 14). An adhesion force of 3 N/cm² is quite significant and is capable of maintaining strong adhesion between the patch and the intestine. This is clear from the fact the mass of a typical patch is 1.2 mg, corresponding to a weight of about 11.2 μN. On the other hand, the adhesion force offered by the mucoadhesive polymer for a 2 mm patch is about 100 mN per patch. Thus, the adhesive force is significantly higher than the inertial forces and should maintain good adhesion between the patch and the intestine. Under in vivo conditions, the patch may experiences forces in addition to its own weight due to peristalsis of the intestine. Accordingly, the difference between the adhesive force and the detachment force may be smaller in vivo. High adhesive forces obtained between the patch and the intestine are attributed to compression of the mucoadhesive layer under high pressure during the fabrication process. This process increases the amount of mucoadhesive material per unit area of the patch compared to that obtained in patches prepared by simple casting process without compression (open bars, P<0.05) (FIG. 14). Adhesion force was independent of compression pressure used in preparation of patches. The measured adhesion forces are generally comparable to those previously measured between mucoadhesive polymer films and buccal membrane. Since there are no standard test methods specifically designed for bioadhesion analysis, it is difficult to quantitatively compare adhesion measurements from different research groups. Furthermore, since the adhesion force is likely to depend on the method of detachment, these measurements should be considered as a preliminary assessment of bioadhesiveness of the patches. In another experiment, adhesion force decreased when higher volume of fluid was present in the lumen (FIG. 13). To explore the relationship between the thickness of water layer on intestinal surface and bioadhesion of the patch, adhesion force of the patch was assessed. Data plotted in FIG. 15 show the decrease of adhesion force with the increase in PBS layer thickness on the intestine surface in both compressed (closed squares) and non-compressed (closed circles) patch systems. For a compressed patch, the adhesion force exceeded 1 N/cm² even when the patch was under a 3 mm layer of PBS, which is equivalent to an approximate PBS volume of 32% in the intestine. This result is comparable with the data obtained from the dynamic experimental system, in which the patches exhibited strong adhesion (˜1.5 N/cm²) even when the intestine was filled with up to 40% of water. It is expected that under typical physiological conditions, the intestine is filled with less than 20% fluids (the percentage was roughly calculated using an intestinal volume of 4000-5000 cm³ and the average fluid volume in the small intestine of 400-800 ml). Therefore, in most typical conditions, the patch could potentially adhere to the intestinal wall.

### EXAMPLE 3

Intestinal delivery of insulin patches in non-diabetic rats: All animal experiments were4conducted under aseptic conditions using institutionally approved protocols. Male Sprague Dawley (SD) rats, weighing 350-450 g fasted for 16 hours were anesthetized using gas anesthesia (1.25-4% isofluorane in oxygen). Rat intestine was exposed through a midline abdominal incision (2.0 cm). A small longitudinal incision (5 mm) was made about 5 cm from the proximal end of the small intestine. Patches (3-6 pieces, 2 mm in diameter) containing insulin (0.4-1.2 IU/patch, totally 5 IU/kg or 10 Iu/kg per rat) were randomly inserted through the opening into the lumen. The incisions were then sealed by surgical tissue (NEXABAND®, Veterinary Products Laboratories, Phoenix, Ariz.). Blood samples (0.1 ml) were collected from the tail vein every 1 hour up to 8 hours after the delivery of patches. Blood glucose level was measured using a glucometer (Excite® XL, Bayer Corporation, Elkhart, Ind.). In one set of experiments, sodium glycocholate (Sigma Chemicals, St. Louis, Mo.) (10 mg/rat) was incorporated together with insulin in the patch to assess whether chemical enhancers could synergistically enhance insulin absorption together with the patch system. For negative control experiments, either insulin solution (10 IU/kg), or blank patches (no insulin) was administered in the intestine. Positive controls were performed by subcutaneously injecting insulin solution (pH 7) (1 IU/kg). Blood glucose values were plotted as a function of time. The areas above the glucose curve (AAC) were calculated by the trapezoidal method (Carino G P, Jacob J S, Mathiowitz E. 2000. Nanosphere based oral insulin delivery. J Controlled Release 65:261-269). The apparent relative pharmacological bioavailability of insulin from non-diabetic rats was calculated by comparing the AAC following intestinal administration under different doses with that following subcutaneous administration.

Due to low permeability and high susceptibility to proteolytic degradation, the absorption of insulin into the blood circulation from the gastro-intestinal tract is generally poor. Several strategies have been proposed to increase oral insulin bioavailibility. With the aid of permeation enhancers, such as bile salts and fatty acids, the permeability of the lipid bilayer of cell membranes of the epithelial cells may be increased (Uchiyama T, Sugiyama T, Quan Y S, Kotani A, Okada N, Fujita T, Muranishi S, Yamamoto A. 1999. Enhanced permeability of insulin across the rat intestinal membrane by various absorption enhancers: their intestinal mucosal toxicity and absorption-enhancing mechanism of n-lauryl-beta-D-maltopyranoside. J Pharm Pharmacol 51:1241-1250; Scott-Moncrieff J C, Shao Z, Mitra A K. 1994. Enhancement of intestinal insulin absorption by bile salt-fatty acid mixed micelles in dogs. J Pharm Sci 83:1465-1469). Moreover, the use of protease inhibitors such as aprotinin, bacitracin and soybean trypsin inhibitor has also been shown to be effective in reducing protein degradation in the intestinal tract (Morishita M, Morishita I, Takayama K, Machida Y, Nagai T. 1993. Sitedependent effect of aprotinin, sodium caprate, Na₂EDTA and sodium glycocholate on intestinal absorption of insulin. Biol Pharm Bull 16:68-72; Yamamoto A, Taniguchi T, Rikyuu K, Tsuji T, Fujita T, Murakami M, Muranishi S. 1994. Effects of various protease inhibitors on the intestinal absorption and degradation of insulin in rats. Pharm Res 11:1496-1500). Other delivery strategies have been primarily focused on utilization of microencapsulation technologies. Micro/nanospheres can protect insulin from enzyme degradation in the intestine, while nanospheres or nanocapsules can further facilitate insulin transport across the epithelia by way of Peyer's patches (Aprahamian M, Michel C, Humbert W, Devissaguet J P, Damge C. 1987. Transmucosal passage of polyalkylcyanoacrylate nanocapsules as a new drug carrier in the small intestine. Biol Cell 61:69-76). Despite significant research in this area, oral delivery of proteins still poses a challenging scientific goal.

Intestinal patches described in this paper offer several advantages over standard oral tablets, sustained release formulations, and microspheres. Specifically, the patches offer high surface area per unit mass of the patch, thereby increasing their adhesion on the intestinal wall. Adhesion of patches on the wall should also localize the drug near the wall thereby offering increased concentration gradient for its transport. The protective layer of the patch also offers two advantages. First, this layer minimizes drug loss into the intestine, thereby forcing the drug to diffuse towards the intestinal wall. Furthermore, this layer also minimizes enzyme penetration into the patch, thereby offering protection for polypeptides drugs like insulin.

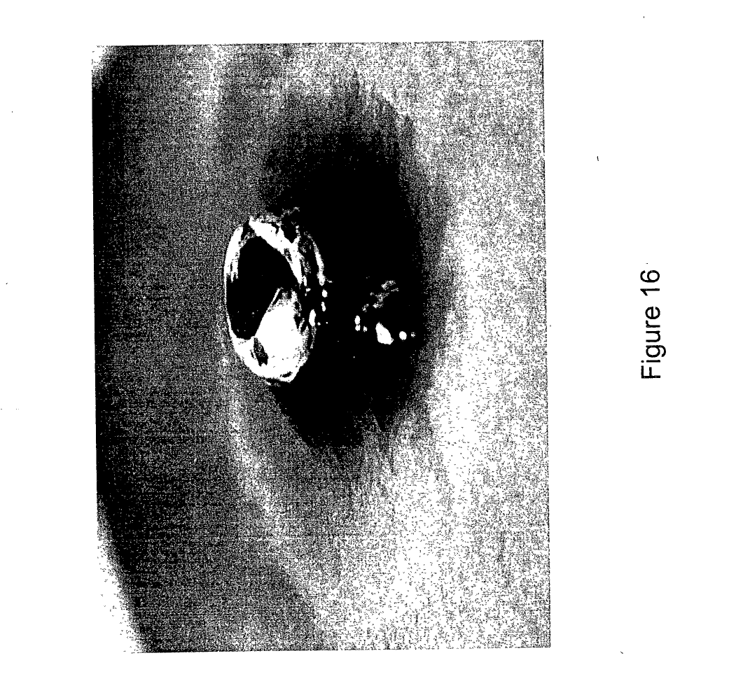

FIG. 16 shows an image of a patch loaded with sulforhodamine B that has adhered to pig intestine for 1 hour. The patch is swollen due to water absorption and has released sulforhodamine B into the intestinal wall. EC layer (visible as a reflective layer at the top) assists in maintaining the integrity of the patch and minimizes sulforhodamine loss into the lumen.

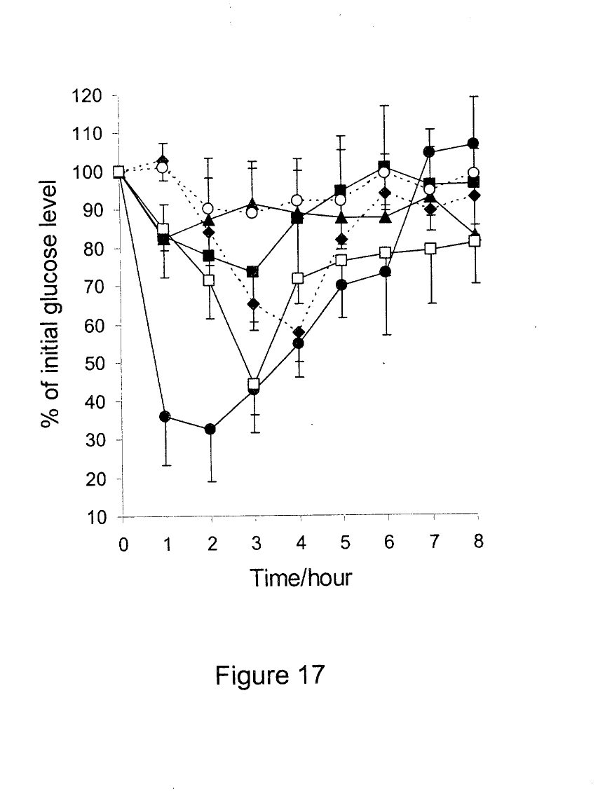

Having established the adhesion of patches on the intestinal wall and unidirectional solute release, we assessed whether these patches can effectively deliver insulin from the intestine in non-diabetic rats. FIG. 17 shows results of these studies when patches were administered intestinally (closed squares correspond to an insulin patch dose of 5 IU/kg and closed diamonds correspond to an insulin patch dose of 10 IU/kg). Open circles show controls where insulin solution (10 IU/kg) was administered in the intestine. As expected, injection of insulin solution in the intestine did not produce detectable hypoglycemia. However, administration of patches containing insulin (5 Iu/kg) decreased blood sugar level from 95±9 mg/dl to 70±12 mg/dl within 2 hours, corresponding to a maximal decrease of 27%. When patches were delivered containing insulin at 10 IU/kg, blood sugar level dropped from 118±15 mg/dl to 67±4 mg/dl within 4 hours (corresponding to a 43% reduction). To quantify relative pharmnacological bioavailability of insulin, rats were injected with 1 U/kg subcutaneous insulin. Results of these experiments are shown by closed circles. Compared to hypoglycemia achieved by subcutaneous injections, relative bioavailability of insulin from patches form was 6.9±2.3% (5 IU/kg) and 4.5±0.9% (10 IJ/kg). Addition of sodium glycocholate (10 mg/rat) to patches further increased the effectiveness of the patches. Specifically, insulin patches at a dose of 5 IU/kg significantly decreased blood glucose level from 124±12 mg/dl to 55±20 mg/dl, with a maximal glucose reduction about 56% in 3 hours. The bioavailability under this condition reached 14.2±1.0% compared to subcutaneous insulin injection of 1U/kg. At the end of the in vivo experiment, the intestine was excised to locate the patches. 8 hours after their insertion in the intestine, some of the patches could be found within 5 cm from the site of their insertion in the intestine.

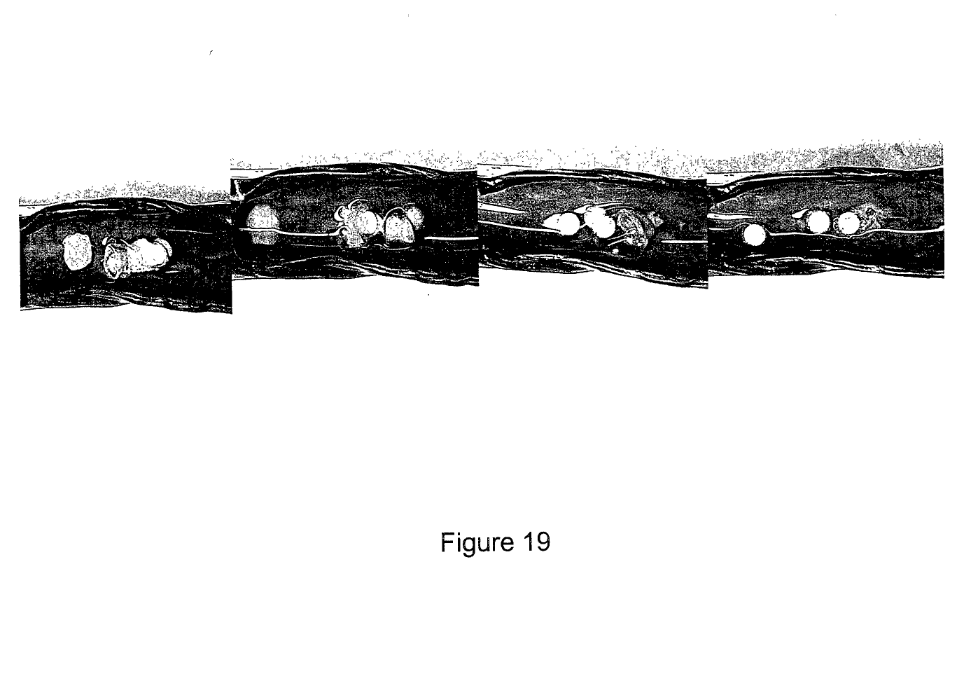

FIG. 19 shows a series of images demonstrating release of patches from a capsule and adhesion of patches on the intestine.

### EXAMPLE 4

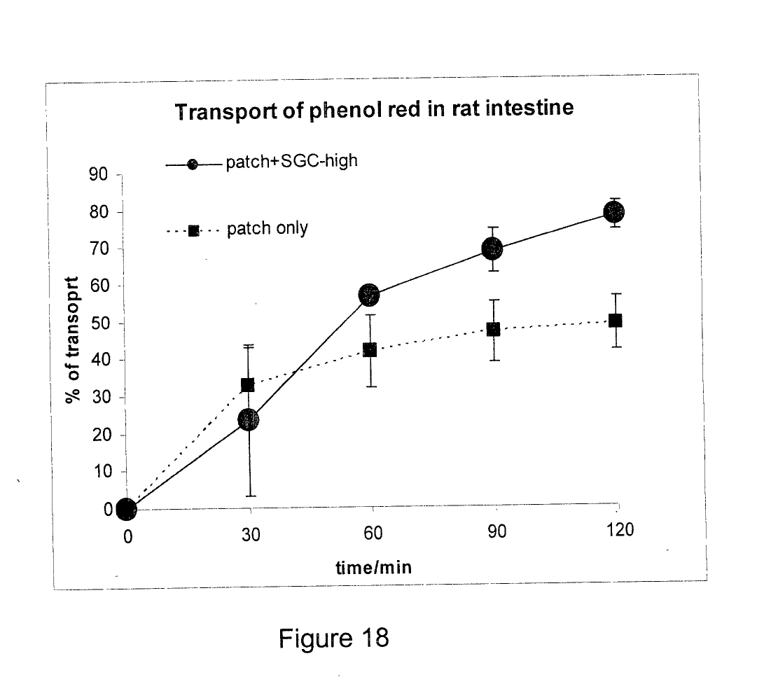

The effectiveness of patches can be improved by further incorporation of chemical enhancers. These enhancers can be selected from a group including but not limited to fatty acids, fatty alcohols, esters, surfactants, and protease inhibitors. FIG. 18 shows the effect of an enhancer, sodium glaucocholate on delivery of phenol red from patches (circles) compared to patches without sodium glaucocholate (squares).

The following references are each incorporated herein by reference: S. Okada, et al., “In vitro evaluation of polymerized liposomes as an oral drug delivery system,” Pharm. Res. 12 (1995), pp. 576-582; H. Chen, V. Torchilin and R. Langer, Polymerized liposomes as potential oral vaccine carriers: stability and bioavailability. J. Controlled Release 42 (1996), pp. 263-272.); Mathiowitz, J.S. Jacob, Y. S. Jong, G. P. Carino, D. Chickering, P. Charturved, C. A. Santos, K. Vijayaraghavan, S. Montogomery, M. Bassett and C. Morrell, Biologically erodable microspheres as potential oral drug delivery systems. Nature 386 (1997), pp. 410-414; N. Santiago, S. Milstein, T. Rivera, E. Garcia, T. Zaidi, H. Hong and D. Bucher, Oral Immunization of rats with proteinoid microspheres encapsulating influenza virus antigens. Pharm. Res. 10 8 (1993); C. Damgé, C. Michel, M. Aprahamian and P. Couvreur, New approach for oral administration of insulin with polyalkycyanoacrylate nanocapsules as drug carrier. Diabetes 37 (1988), pp. 246-251; Carino GP, Jacob J S, Mathiowitz E. Nanosphere based oral insulin delivery. J Control Release 2000 Mar 1;65(1-2):261-9; A. M. Hillery, I. Toth and A. T. Florence, Co-polymerised peptide particles II: Oral uptake of a novel copolymeric nanoparticle delivery system for peptides. J. Controlled Release 42 (1996), pp. 65-73; H. Chen, V. Torchilin and R. Langer, Lectin-bearing polymerized liposomes as potential oral vaccine carriers. Pharm. Res. 13 9 (1996), pp. 1378-1383; Kawashima Y, Yamamoto H, Takeuchi H, Kuno Y. Mucoadhesive DL-lactide/glycolide copolymer nanospheres coated with chitosan to improve oral delivery of elcatonin. Pharm Dev Technol 2000;5(1):77-85; and Lim S T, Martin G P, Berry D J, Brown M B. Preparation and evaluation of the in vitro drug release properties and mucoadhesion of novel Yamamoto A. 1999. Enhanced permeability of insulin across the rat intestinal membrane by various absorption enhancers: their intestinal mucosal toxicity and absorption-enhancing mechanism of n-lauryl-beta-D-maltopyranoside. J Pharm Pharmacol 51:1241-1250, Yamamoto A, Okagawa T, Kotani A, Uchiyama T, Shimura T, Tabata S, Kondo S, Muranishi S. 1997. Effects of different absorption enhancers on the permeation of ebiratide, an ACTH analogue, across intestinal membranes. J Pharm Pharmacol 49:1057-1060, Marschuitz M K, Bemkop-Schnüirch A. 2000. Oral peptide drug delivery: polymerinhibitor conjugates protecting insulin from enzymatic degradation in vitro. Biomaterials 21:1499-1507, Marschutz M K, Puttipipatkhachom S, Bernkop-Schnurch A. 2001. Design and in vitro evaluation of a mucoadhesive oral delivery system for a model polypeptide antigen. Pharmazie 56:724-729, Ma X Y, Pan G M, Lu Z, Hu J S, Bei J Z, Jia J H, Wang SG. 2000. Preliminary study of oral polylactide microcapsulated insulin in vitro and in vivo. Diabetes Obes Metab 2:243-250, Damge C, Vranckx H, Balschmidt P, Couvreur P. 1997. Poly(alkyl cyanoacrylate) nanospheres for oral administration of insulin. J Pharm Sci 86:1403-1409, Carino G P, Jacob J S, Mathiowitz E. 2000. Nanosphere based oral insulin delivery. J Controlled Release 65:261-269, Ogiso T, Funahashi N, Tsukioka Y, Iwaki M, Tanino T, Wada T. 2001. Oral delivery of synthetic eel calcitonin, elcatonin, in rats. Biol Pharm Bull 24:656-661, Dogru S T, Calis S, Oner F. 2000. Oral multiple w/o/w emulsion formulation of a peptide salmon calcitonin: in vitro-in vivo evaluation. J Clin Pharm Ther 25:435-443, Iwanaga K, Ono S, Narioka K, Kakemi M, Morimoto K, Yamashita S, Namba Y, Oku N. 1999. Application of surface-coated liposomes for oral delivery of peptide: effects of coating the liposome's surface on the GI transit of insulin. J Pharm Sci 88:248-252, Kisel M A, Kulik L N, Tsybovsky I S, Vlasov AP, Vorob'yov M S, Kholodova E A, Zabarovskaya Z V. 2001. Liposomes with phosphatidylethanol as a carrier for oral delivery of insulin: studies in the rat. Int J Pharm 216:105-114, Bernkop-Schnurch A, Apprich I.. 1997. Synthesis and evaluation of a modified mucoadhesive polymer protecting from α-chymotrypsinic degradation International Journal of Pharmaceutics 146: 247-254, Scott-Moncrieff J C, Shao Z, Mitra A K. 1994. Enhancement of intestinal insulin absorption by bile salt-fatty acid mixed micelles in dogs. J Pharm Sci 83:1465-1469, Morishita M, Morishita I, Takayama K, Machida Y, Nagai T. 1993. Sitedependent effect of aprotinin, sodium caprate, Na₂EDTA and sodium glycocholate on intestinal absorption of insulin. Biol Pharm Bull 16:68-72, Yamamoto A, Taniguchi T, Rikyuu K, Tsuji T, Fujita T, Murakami M, Muranishi S. 1994. Effects of various protease inhibitors on the intestinal absorption and degradation of insulin in rats. Pharm Res 11:1496-1500, Aprahamian M, Michel C, Humbert W, Devissaguet J P, Damge C. 1987. Transmucosal passage of polyalkylcyanoacrylate nanocapsules as a new drug carrier in the small intestine. Biol Cell 61:69-76, Peh K K, Wong C F. 1999. Polymeric films as vehicle for buccal delivery:swelling, mechanical, and bioadhesive properties. J Pharm Pharmaceut Sci 2:53-61, Tiwari D, Goldman D, Sause R, Madan P L. 1999. Evaluation of polyoxyethylene homopolymers for buccal bioadhesive drug delivery device formulations. AAPS PharnSci 1:E13, Wong C F, Yuen K H, Peh K K. 1999. An in-vitro method for buccal adhesion studies: importance of instrument variables. Int J Pharm 180:47-57, Peppas N A, Sahlin J J. 1996. Hydrogels as mucoadhesive and bioadhesive materials: a review. Biomaterials 17:1553-1561, Lowman A M, Morishita M, Kajita M, Nagai T, Peppas N A. 1999. Oral delivery of insulin using pH-responsive complexation gels. J Pharm Sci 88:933-937.

What is claimed is:

1. A method of delivery of at least one active agent to an organism comprising;
a) encapsulating at least one active agent into at least one patch comprising at least one mucoadhesive side and one poorly permeable side.
b) placing the patches in a containment.
c) releasing the patches from the containment within the body.

2. Method of claim 1, wherein the containment is coated with a material showing pH dependent solubility.

3. Method of claim 1 further comprising addition of at least one filler material to the containment.

4. Method of claim 1, wherein the patches have one dimension between 100 micrometer and 5 millimeter and two dimensions of between 100 micrometer and 2 millimeter.

5. Method of claim 1, wherein the patch has at least one substantially permeable side and at least one substantially impermeable side.

6. Method of claim 1, wherein the mucoadhesive side is composed of materials selected from the Carbopol, pectin, and sodium carboxylmethylcellulose (SCMC).

7. Method of claim 1 wherein the poorly permeable material is ethylcellulose or poly(lactic co-glycolic acid).

8. Method of claim 1 wherein encapsulation of active agents is performed by making a homogeneous mixture of therapeutic agent and the mucoadhesive material.

9. Method of claim I wherein encapsulation of therapeutic agents is performed using microspheres of a biocompatible material.

10. Method of claim I wherein the active agent is a therapeutic drug selected from a group of proteins, peptides, vaccines, small molecules, and polysaccharides.

11. Method of claim I wherein a permeability enhancer is included in the patch.

12. Method of claim 1 wherein a protease inhibitor is included in the patch.

13. Method of claim I wherein the containment swallowed orally for release of patches in the stomach, small intestine, large intestine, or colon.

14. Method of claim 1, wherein the containment is dissolved in the oral cavity for release of patches in mouth

15. Method of claim 1, wherein the containment is delivered rectally for release of patches near colon.

16. A device for delivering active agents to an organism comprising:
a) at least one patch containing at least one active agent and possessing at least one mucoadhesive side and one poorly permeable side.
b) a containment to encapsulate the patches.

17. Device of claim 16 wherein the containment is a tablet.

18. Device of claim 16 wherein the containment in a capsule.

19. Device of claim 16 wherein the containment is coated with material that is designed to dissolve the containment in the intestine

20. Device of claim 16 wherein the material used for coating the containment is a pH sensitive material that dissolves at a pH greater than 6.

21. Device of claim 16 wherein the material used for coating the containment is designed to dissolve in the colon.
